(19) United States
(12) Patent Application Publication
(10) Pub. No.: US 2014/0316262 A1
(43) Pub. Date: Oct. 23, 2014

(54) PREOPERATIVE IDENTIFICATION OF PERFORATOR VESSELS IN FLAPS TO BE USED IN RECONSTRUCTIVE SURGERY

(71) Applicant: Jerilee A. HAVENS, Wilmington, NC (US)

(72) Inventor: Jerilee A. HAVENS, Wilmington, NC (US)

(73) Assignee: NOVADAQ TECHNOLOGIES INC., Mississauga (CA)

(21) Appl. No.: 13/542,537

(22) Filed: Jul. 5, 2012

Related U.S. Application Data
(63) Continuation of application No. 12/391,609, filed on Feb. 24, 2009, now abandoned.
(60) Provisional application No. 61/031,520, filed on Feb. 26, 2008.

(51) Int. Cl. A61B 6/00

(52) U.S. Cl. CPC A61B 6/481
USPC 600/431

(57) ABSTRACT

A method is disclosed for preoperative identification of a perforator vessel in a flap to be used in reconstructive surgery. The tissue area suitable for a flap is identified by injecting a bolus of ICG into the bloodstream, illuminating the tissue area with excitation light and observing ICG fluorescence through the skin. The location of the perforator vessel in the tissue area is marked on the patient's skin. An incision is made in the tissue area and the flap is harvested only after a suitable perforator vessel has been identified.

### CROSS-REFERENCE TO RELATED APPLICATIONS

This application claims the benefit of U.S. Provisional Application Ser. No. 61/031,520, filed Feb. 26, 2008, the entire content is incorporated herein by reference.

### BACKGROUND OF THE INVENTION

Abdominal donor-site flaps have become the standard for autologous breast reconstruction since the early 1980s. Within the abdomen, free flap options range from complete transverse rectus abdominis musculocutaneous (TRAM) flaps to isolated perforator flaps, such as the deep inferior epigastric artery (DIEA) perforator flap. Perforator flaps have allowed a reliable transfer of the patient's own skin and fat also in other areas of tissue reconstruction, with minimal donor-site morbidity. Flaps that relied on a random pattern blood supply were soon supplanted by pedicled, axial patterned flaps that could reliably transfer great amounts of tissue. The advent of free tissue transfer allowed an even greater range of possibilities to appropriately match donor and recipient sites. The increased use of perforator flaps has escalated the need for a pre-operative familiarity of an individual's particular anatomical feature of the flaps and its perforating branches, particularly given the significant variation in that anatomy of the vascular supply to the abdominal wall.

Conventionally, the standard for preoperative imaging of the DIEA flap, particularly the TRAM flaps, has been Doppler ultrasonography, which has been used extensively for TRAM flap selection and mapping of perforators before DIEA perforator flaps. Significant inconsistencies between the operative findings and the results of Doppler ultrasound are not uncommon, and the presentation of ultrasonographic findings to the surgeon is not optimal. The search for a more favorable imaging modality is thus continuing, with recent interest in the use of indocyanine green (ICG) fluorescence imaging, wherein blood circulation is assessed through the skin on the basis of a fluorescence signal. Fluorescence in ICG with an emission peak around 830 nm occurs as a result of excitation by radiation in the near-infrared spectral range. Excitation light with a wavelength around 800 nm can be produced, for example, by a diode laser, light emitting diodes (LED), or other conventional illumination sources, such as arc lamps, halogen lamps with a suitable bandpass filter. The skin is transparent to this wavelength.

ICG strongly binds to blood proteins and has previously been used for cardiac output measurement, hepatic function evaluation, and ophthalmic angiography, with few adverse reactions. In previously reported uses for assessing the patency of perforator vessels in donor-site flaps, ICG videoangiography was performed after partial dissection of the flap, the unit being still in the abdominal region or after transposing the flap to the mammary region, to identify and intraoperatively mark areas of the skin ellipse which are not sufficiently fluorescent and therefore not sufficiently vascularized. These areas are then removed.

ICG fluorescence imaging has previously also been successfully used to assess and validate the anastomotic patency and the arterial and venous flow to prevent post-operative flap and graft failure.

However, there is still a need for preoperatively identifying the location and optionally dimensions of perforator vessels in perforator flaps to be transplanted by a simple, accurate and non-invasively method, before any incision is made.

### SUMMARY OF THE INVENTION

According to one aspect of the invention, a method for preoperative identification of a perforator vessel in a flap to be used in reconstructive surgery is disclosed, which includes the steps of locating a tissue area suitable for a flap, injecting a bolus of ICG into the bloodstream, illuminating the tissue area with excitation light and observing ICG fluorescence through the skin, and marking the location of the perforator vessel on the patient's skin in the tissue area. All the above steps are carried out before any incision is made in the tissue area.

In one embodiment, after the location of the perforator vessel has been marked on the patient's skin, the tissue area with the marked perforator vessel(s) may be incised, and the flap may be harvested and transplanted to the diseased or traumatized area to be reconstructed.

In another embodiment, a bolus of ICG may be injected intravenously into the bloodstream to evaluate perfusion of the perforator vessel(s) in the harvested flap. The anastomotic patency and the arterial and venous flow after reconstruction may finally be evaluated by injecting yet another bolus of ICG into the bloodstream so as to prevent post-operative flap and graft failure.

### BRIEF DESCRIPTION OF THE DRAWINGS

FIG. 1 shows schematically a camera system for observing ICG fluorescence;

FIG. 2 shows general line of anatomical landmarks, with a marking showing the perforator location, based on observation of ICG fluorescence with a camera of the type shown in FIG. 1; and

FIG. 3 is an image of the transplanted flap.

### DETAILED DESCRIPTION

The invention is directed to preoperative determination of the location of perforator vessels in perforator flaps by a non-invasive method, before any incision is made.

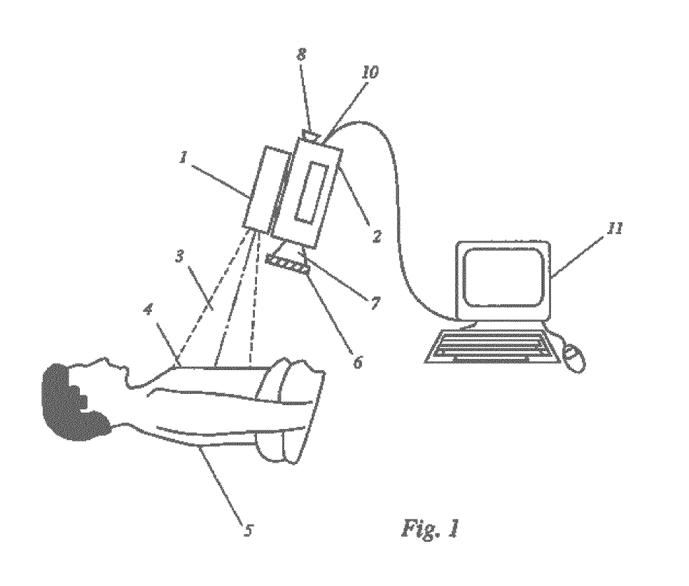

FIG. 1 shows schematically a device for non-invasively determining through the skin tissue perfusion in operative, in particular preoperative, applications by ICG fluorescence imaging. An infrared light source, for example, one or more diode lasers or LEDs, with a peak emission of about 780-800 nm for exciting fluorescence in ICG is located inside housing 1. The fluorescence signal is detected by a CCD camera 2 having adequate near-IR sensitivity; such cameras are commercially available from several vendors (Hitachi, Hamamatsu, etc.). The CCD camera 2 may have a viewfinder 8, but the image may also be viewed during the operation on an external monitor which may be part of an electronic image processing and evaluation system 11.

Expanded laser light 3 emerges from the housing 1 to illuminate an area of interest 4, i.e. the area where a flap with suitable perforator vessels is expected to be located. The area of interest may be about 10 cm×10 cm, but may vary based on surgical requirements and the available illumination intensity and camera sensitivity.

A filter 6 is typically placed in front of the camera lens 7 to block excitation light from reaching the camera sensor, while allowing fluorescence light to pass through. The filter 6 may be an NIR long-wave pass filter (cut filter), which is only transparent to wavelengths greater than about 815 nm, or preferably a bandpass filter transmitting at peak wavelengths of between 830 and 845 nm and having a full width at half maximum (FWHM) transmission window of between about 10 nm and 25 nm, i.e. outside the excitation wavelength band. The camera 2 may also be designed to acquire a color image of the area of interest to allow real-time correlation between the fluorescence image and the color image.

In the context of the present invention, the device illustrated in FIG. 1 can be used to:

1. Identify/locate perforator vessels—this will assist the surgeon in selecting the best flap or flap zone for use during the reconstruction.

2. Validate anastomotic patency and arterial and venous flow—this can potentially improve outcomes to eliminate flap failure which can be a result of poor arterial flow and inadequate perfusion as well as poor venous return resulting in congestion.

3. Visualize and confirm complete tissue perfusion—micro-vascular perfusion to the entire flap and native tissue is critical to flap survival.

As an important aspect of the invention, suitable perforator vessels are identified with high accuracy and non-invasively, before the surgeon makes any incision, so as to reduce unnecessary trauma.

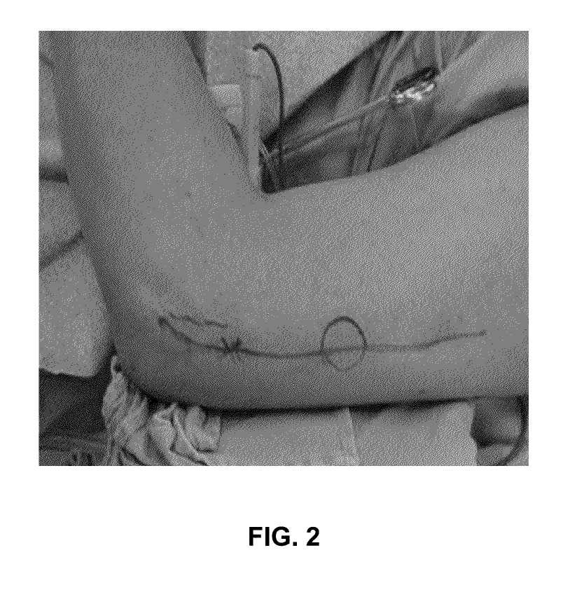

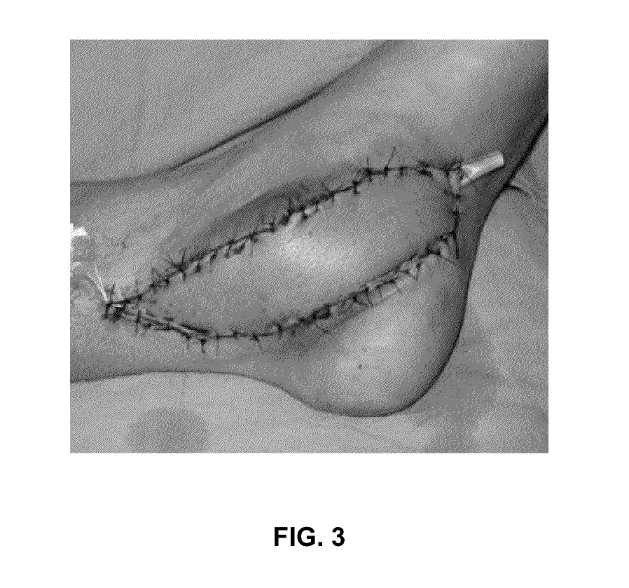

FIG. 2 shows an image of a patient's arm from which a flap is to be removed for reconstruction of the patient's foot, shown in FIG. 3. The general area where perforator vessels are generally found is indicated with a line of anatomical landmarks. The same area is then illuminated with the excitation light 3 and the ICG fluorescence is observed with the camera 2, shown in FIG. 1. Prior to making an incision, (after the patient is prepped and draped), the surgeon will inject 10 mg of ICG and watch for the first blush of fluorescence coming into the region of the “pre-marked flap” on the skin. The surgeon can circle (see circle surrounding the line in FIG. 1) these “designated areas” of high fluorescence or a specific point of fluorescence and note how the areas play into the overall flap. Based on the ICG fluorescence information, the surgeon can then change the original outline of his flap to now incorporate these areas that may be different than what was originally thought to be the size and dimensions of the flap.

This allows the surgeon to effectively change the operative plan and impact decision making, both pre-operatively and intra-operatively. For example:

A. Surgeons may decide to change the actual size and location of the flap-prior to making an incision based on the location of the perforators.

B. Surgeons may change the actual procedure from free flap to pedicled Flap—if good perforators for a bilateral flap cannot be identified.

C. Surgeons may change the site of flap harvest, for example, from the right side to the left side, and vice versa.

1. For unilateral breast reconstruction, some surgeons prefer contralateral flaps, but if one side of the abdomen shows better perfusion, they can:
a. enlarge the size of the contralateral flap to include the area of perfusion visualized during ICG fluorescence imaging,
b. take the flap from the ipsilateral side, making the arterial and venous pedicle longer to make the anastomosis of the artery and vein easier, or
c. change the size of the flap to accommodate anastomosis to the thoracodorsal artery versus the internal mammary artery.

During a unilateral DIEP flap procedure making use of an abdominal flap, ICG fluorescence imaging will not only be able to locate the best perforators, but may change which side of the abdomen is most suitable for harvest. This may save operative time, for example, if there appears to be no really good perfusion or perforator on one particular side, then the surgeon can turn to the other side of the abdomen and begin the operation there. ICG fluorescence imaging not only effectively affects the surgeon's decision about the size and location of the flap, but may reduce the operating time by decreasing the time that would need to be spent to locate the best perforators on both sides of the flap. One side may yield better perforators than the other.

Conversely, if a flap that was intended for use, for example, based on Doppler sonography, now shows reduced ICG fluorescence, this may effectively change the overall operation strategy. For example, if the intent was to perform a bilateral free flap and the flap area exhibits poor perfusion, then the operation may be changed from a free flap to a pedicled flap, saving valuable time and potential post-operative flap failure. Locating perforators prior to the operation may save time and improve outcomes when rotating a skin flap for a head and neck procedure where they must use tissue expanders. Realizing after the tissue is expanded that there is no good perforator in the flap may lead to diminished expectations and more severe emotional and/or psychological issues.

Using pre-operative ICG fluorescence imaging to identify flaps with suitable perforator vessels may reduce operative procedural times. The ability to simply and efficiently locate perforator vessels and assess the quality of perforators from one side of the proposed flap versus another can save valuable time. Dissection of one side of the abdomen, and finding perforators manually can take 1 ½ hours to 2 hours or more. Operating room time is expensive so a reduction in the time to accurately locate perforators could result in significant cost reduction.

Using pre-operative ICG fluorescence imaging to identify flaps with suitable perforator vessels may enhance or improve surgical outcomes, because adequate perfusion in the entire flap may change the size or length of the pedicle either while in situ or after the anastomosis has been performed.

ICG fluorescence imaging can subsequently be applied intra-operatively, i.e., after the flap is dissected, gives the surgeon the ability to see the amount of perfusion in the flap in situ. As previously described for the preoperative stage, 10 mg of ICG is injected intravenously and the amount of arterial inflow is imaged first, thereafter panning across the flap and noting the perfusion in the skin flap all the way out to the tip of the flap. If it appears that the tip of the flap is not well perfused, the flap can be reshaped to exclude this portion of the flap prior to use.

Once the flap is inset (sewn in place), perfusion is once more checked with ICG fluorescence imaging and the amount of fluorescence in both the native tissue and the flap tissue is compared. If any portion of the tissue appears darker or does not seem to fluoresce, this may denote potentially decreased perfusion, suggesting revision of the graft before the patient leaves the operating room.

ICG fluorescence imaging may be particularly useful in determining if an abdominal flap can be supported by the SIEA (Superficial Inferior Epigastric Artery). This is important for at least the following reasons:

1. The procedure is less time consuming than TRAM or DIEP.

2. A SIEA flap has a potential to eliminate abdominal donor site morbidity because the muscle is not actually incised

3. The SIEA is currently not frequently used; but with ICG fluorescence imaging able to show intraoperative perfusion of SIEA flaps in situ, the more intensive DIEP flap may no longer be used.

4. Shorter hospital stay

5. Similar aesthetic results

6. No injury to the anterior rectus sheath or underlying muscles

The described embodiments detect a fluorescence signal emitted transcutaneously by ICG following excitation in the near-infrared spectral range. However, those of skill in the art will appreciate that other dyes which can be excited and emit fluorescence in a spectral range where tissue transmits light can also be used.

While the invention is receptive to various modifications, and alternative forms, specific examples thereof have been shown in the drawings and are herein described in detail. It should be understood, however, that the invention is not limited to the particular forms or methods disclosed, such as the reconstruction of a patient's foot, and that the method of preoperatively identifying suitable perforator flaps for tissue grafting can be applied to breast reconstruction, reconstruction of complicated postoncologic defects in the head and neck region, traumatic injury, and the like. The invention is hence meant to cover all modifications, equivalents, and alternatives falling with the spirit and scope of the appended claims.

What is claimed is:

1. A method for preoperative identification of a perforator vessel in a flap to be used in reconstructive surgery in a human patient using indocyanine green fluorescence angiography, comprising the steps of:
before making an incision in the human patient, locating a tissue area suitable for a flap;
injecting a bolus of indocyanine green into a bloodstream of the patient;
illuminating the tissue area with excitation light and observing indocyanine green fluorescence through the patient's skin; and
marking a location of the perforator vessel on the patient's skin in the tissue area based upon indocyanine green fluorescence, thereby improving surgical outcomes.

2. The method of claim 1, further comprising the steps of:
incising the tissue area comprising the perforator vessel whose location has been marked on the patient's skin;
harvesting the flap; and
transplanting the flap to a diseased or traumatized area to be reconstructed.

3. The method of claim 2, further comprising injecting a second bolus of indocyanine green intravenously into the bloodstream to evaluate perfusion of the perforator vessel in the harvested flap.

4. The method of claim. 2, and claim 3, further comprising injecting a third bolus of indocyanine green into the bloodstream after transplanting to evaluate anastomotic patency and arterial and venous flow to prevent post-operative flap and graft failure.

5. A method of assessing perfusion in a tissue flap used in reconstructive surgery in a patient, comprising the steps of:
incising a tissue area comprising at least one perforator vessel whose location has been confirmed using indocyanine green fluorescence angiography;
harvesting the tissue flap;
injecting a bolus of indocyanine green into a bloodstream of the patient;
illuminating the tissue area with excitation light and observing indocyanine green fluorescence through the patient's skin; and
assessing perfusion in the tissue flap on the basis of observed indocyanine green fluorescence, thereby improving surgical outcomes.
